(19) United States
(12) Patent Application Publication
(10) Pub. No.: US 2004/0265904 A1
(43) Pub. Date: Dec. 30, 2004

(54) Diagnostic method

(76) Inventors: Michael James Stack, Surrey (GB); Melanie Jane Chaplin, Surrey (GB); Jemma Clark, Surrey (GB)

Correspondence Address: NIXON & VANDERHYE, PC, 1100 N GLEBE ROAD, 8TH FLOOR, ARLINGTON, VA, 22201-4714, US

(21) Appl. No.: 10/493,572

(22) PCT Filed: Oct. 23, 2002
(86) PCT No.: PCT/GB02/04789

(30) Foreign Application Priority Data
Oct. 25, 2001 (GB) 0125606.4

(51) Int. Cl. G01N 33/53

(52) U.S. Cl. 435/7.1

(57) ABSTRACT

A method for typing a strain of a transmissible spongiform encephalophathy (TSE) in an infected animal, said method comprising: a) separating a sample of abnormal prion protein on the basis of molecular weight and/or glycoform ratios, and detecting the separated forms; b) detecting in the sample the presence of a peptide sequence, wherein the presence of said peptide sequence within abnormal prion protein is capable of distinguishing a particular strain of TSE from others, and c) using the results of (a) and (b) to determine the type of TSE strain present in the sample. The method may be used in particular to distinguish BSE from scrapie in sheep.

The present invention relates to a method of typing strains or forms of transmissible spongiform encephalopathies or prion disease found in infected animals, as well as to diagnostic kits and reagents used in the method. In particular, the applicants have found that the method provides a technique for distinguishing between experimentally transmitted BSE in sheep and natural scrapie in sheep.

The transmissible spongiform encephalopathies (TSEs) comprise a group of progressive neurological disorders characterised by neuroparenchymal vacuolation and accumulation of a disease specific isoform of a host coded cell surface sialoglycoprotein called prion protein (PrP). Scrapie, bovine spongiform encephalopathy (BSE) and variant Creutzfeldt-Jakob disease belong to this group of disorders. The diseases appear in various forms or strains.

Already, numerous TSE isolates (usually referred to as strains) have been identified following transmission of a range of sources into rodents. BSE has been transmitted to sheep by oral challenge with as little as 0.5 g of brain material (Foster et al., Vet Rec (1993) 133:339-341). The possibility that some sheep may be naturally infected with the BSE agent is of human and animal health concern.

Currently the only reliable method of TSE agent strain typing is based on the biological properties of an isolate following serial transmission in mice (Bruce et al., 1994, Transmission of bovine spongiform encephalopathy and scrapie to mice: strain variation and the species barrier. Philosophical Transactions-Royal Society of London. Series B. 343, 405-411). However, such methods are extremely time consuming and not all scrapie strains are readily transmitted to mice. Several studies have shown that the patterns of PrP deposition in the brains of mice with scrapie are strain specific (Bruce M E et al. 1989,. Neuroscience Letters 102, 1-6;Bruce 1996, Strain typing studies of scrapie and BSE. In Prion Diseases ed. Baker, H. F. and Ridley, R. M. pp. Totowa/N.J. 07512: Humana Press Inc).

Several other methods have been studied in the hope of providing a more rapid answer to strain identification. All of these are based on properties of disease specific protease resistant fragments of PrP (PrP^{res}) such as the molecular weight (Parchi et al., 1996, Annals of Neurology 39, 767-778), ratio of glycoforms of the PrP^{res} fragments (Collinge et al., 1996, Nature 383, 685-690; Kuczius et al., 1998, Journal of Infectious diseases 178, 693-699; Somerville et al., 1997a, Nature 386, 564-564) or relative protease resistance of PrP^{res} (Kuczius and Groschup, 1999. Molecular Medicine 5, 406-418).

For example, one way of detecting the PrP^{Sc} is by the application of polyacrylamide gel electrophoresis (Laemmli U. K. (1970, Nature 277:680-685) followed by Western Immunoblotting (Towbin H. et al., Proc. Nat. Acad. Sci. USA, 76:4350-4354).

However, these properties of PrP^{res} overlap when different strains or isolates are compared and so they cannot yet be used for definitive strain typing. A conformation assay of PrP^{res} has been described and may provide a means of strain typing but the usefulness of this technique has not yet been established (Safar et al., 1998, Nature Medicine 4, 1157-1165).

The applicants have found that the detection of specific PrP epitopes may provide a useful addition to existing typing methods based upon molecular weights and/or glycoform ratios, which are not always able to provide a reliable distinction, in particular in the case of types of TSE found in sheep.

According to the present invention there is provided a method for typing a strain of a transmissible spongiform encephalopathy (TSE) in an infected animal, said method comprising

a) separating a sample of abnormal prion protein on the basis of molecular weight and/or glycoform ratios, and detecting the separated forms;

b) contacting the sample with an antibody or a binding fragment thereof which binds prion protein from a strain of TSE as found in the sample with a different and distinguishable binding affinity to that of at least one other strain of TSE, and detecting bound antibody or binding fragment; and

c) using the results of (a) and (b) to determine the type of TSE strain present in the sample.

Using this combination or “hybrid” technique, the applicants have found that typing of TSEs may be enhanced, even for example where the prion proteins of the strains have similar molecular weight and/or glycoform ratios.

The antibody or binding fragment thereof will suitably bind a peptide sequence which constitutes an epitopic region of a prion protein of a particular strain. Similar epitopic regions in other prion proteins may have slightly different sequences, which has an effect on the binding of the antibody, or on the way that the protein is affected by the sample preparation in which the abnormal prion protein is separated from the biological material.

As used herein, the term “peptide sequence” refers to sequences, which are in the form of discrete peptides in isolation, or as part of a protein or truncated protein.

Step (b) is suitably effected upon separated material on a gel using a technique such as Western blotting, in which the bound antibody is visualised, for example with a dye. The antibody or binding fragment thereof used is contacted with the separated sample on the gel, and then visualised to produce a signal which has a different intensity depending upon the affinity of the binding. For instance, the antibody or binding fragment has a different and distinguishable affinity for a particular sequence found in a prion protein of one species, such as scrapie, as compared to a similar or corresponding sequence found in another, such as BSE, in the form in which it is present in the sample, for instance a homomgenate which has been treated with a proteinase enzyme.

Differential affinity is suitably judged by a visual comparison of the results although if required, optical equipment as is known in the art may be used to detect the intensity of the signal provided by the bound and visualised antibody. In particular, it is preferred that step (a) of the method of the invention comprises separating processed brain tissue on the basis of molecular weight, for example on a gel, and thereafter detecting proteins for example using an antibody or binding fragment thereof, which binds prion protein, also in a Western blotting technique. In this way, similar blots can be used for both step (a) and step (b), and the diffential binding of step (b) becomes clear.

Measurements by molecular weight and by signal strength of each of the three protein bands which make up the PrPSc (glycoform ratio) is made using computer analysis software. For the former, the software is set to measure the standard molecular weight markers and then gives a calculation of the molecular weights found for the particular sample you are examining. For the glycoform ratio the density of signal for all three bands is considered to be 100% and some differentiations can be made by plotting the percentage signal for the diglycosylated protein band (top band) against the percentage signal from the monoglycosylated protein band (middle band). The relative positions of the plotted points on the graph may then give an indication of PrPSc origin.

Similar methods to that of step (a) alone have been attempted previously to detect differences in glycoform ratios and molecular weights. It is known for example that constituent forms of PrP^{Sc} can be separated by the relative amounts and molecular weights of the di-glycosylated, mono-glycosylated and unglycosylated forms of the protein, using polyacrylamide gel electrophoresis. These were subsequently detected by Western immunoblotting using antiserum produced against PrP. Glycoform ratios and molecular weights have been indicated as being characteristic of particular strains of TSE.

For example, similarities in these factors have supported the likelihood of a bovine origin for vCJD human infections, since those derived from sporadic or iatrogenic forms of CJD are quite different from those of vCJD and BSE (Collinge J. et al. Nature (1996) 383:685-690; Hill A. F. et al., Nature (1997) 389, 448-450).

Attempts to distinguish natural sheep scrapie from sheep experimentally infected with the BSE strain by molecular analysis have also been previously reported, but have led to conflicting results. In one publication (Hill A F, et al., (1998) Neuroscience Letters 255: 159-162) it was reported that a lower unglycosylated band was found in BSE infected sheep in comparison to natural scrapie cases, but in the other two publications (Hope J, et al., (1999) Gen. Vir. 80: 1-4, Hope J, et al., (2000) Corrigendum 81: 1-4) a lower unglycosylated band was found in seven out of eight natural scrapie cases when compared with BSE in sheep samples. Furthermore, in the latter reports, the authors recorded that BSE in sheep showed similar molecular features with an experimental scrapie strain, CH1641. This strain had been propagated in sheep and was originally isolated from a British Cheviot sheep in 1971 (Foster J D, et al., (1988) Vet. Rec. 123: 5-8).

The applicants have found that the molecular weight of the unglycosylated protein band and glycoform ratios for experimental BSE in sheep samples were distinguishable from the natural scrapie and BSE samples (see Example 1 hereinafter) using this method. This method however requires that all the strains are tested in parallel. In view of the complex nature and overlapping nature of the results, it is difficult to use this method alone as a reliable diagnotic test.

However, in those cases, clearer distinction between strains was possible using antibodies which had strong affinity to sequences present in scrapie PrP^{Sc} and greatly reduced affinity to bovine PrP^{Sc}. A particular antibody which achieved this was an antibody raised to a peptide corresponding to amino acids 84-105 of the prion protein or an epitopic region thereof (for example, a peptide corresponding to amino acids 89-104 of sheep).

This was best illustrated by probing the separated forms both with an antibody or binding fragment thereof which showed differential affinity for the two strains as described above, and with an antibody or binding fragment thereof which binds, preferably with a strong affinity, all strains, and comparing the results.

In a particular embodiment, the method of the invention therefore comprises the steps of centrifuging a sample of homogenised tissue from an animal suspected of having a TSE, subjecting the product to an enzyme which digests normal protein, but to which abnormal prion protein is resistant, (such as Proteinase K) separating the thus formed mixture on a gel, probing the separated mixture with (i) an antibody or binding fragment thereof which is specific for a prion peptide, and (ii) antibody or binding fragment thereof which has strong affinity for prion peptides derived from a strain of TSE and weaker affinity for prion peptides derived from other strains of TSE, and typing the strain of TSE on the basis of the characteristics of the signals produced.

For example, this method can be used to detect BSE in experimentally infected sheep, wherein the antibody used in step (i) is sequence an antibody which binds the bovine PrP protein at the amino acid positions 144-152 and the antibody used in (ii) is an antibody which recognises the amino acid sequence in the ovine PrP protein amino acid positions 89-104.

Specifically in this case, if the antibody of (i) doesn't bind then no molecular weights can be measured. If it binds weakly to the antibody of (ii) (such as mAb P4) in comparison to a strong signal by the antibody of (i) (such as mAb 6H4), then the molecular weight differences, and the glycoform ratio of this sample would also be taken into consideration in diagnosing a possible BSE in sheep suspect case. There can therefore be a visual comparison for the different antibody affinities with overall results for all three criteria being used to make a final judgement.

A panel of ruminant brain tissue was subjected to a Western immunoblotting technique and the gels probed using a monoclonal antibody which recognised prion protein. It was found that Romney sheep with ARQ/ARQ genotype and Cheviot sheep with the AHQ/AHQ genotype experimentally infected with BSE give molecular weight values which are more like that obtained for cattle BSE than for ovine scrapie. The primary difference is associated with the unglycosylated protein band which is consistently lower for the BSE in sheep samples than for ovine scrapie cases. This is a similar finding to that found for French experimental BSE in sheep (Baron TGM, et al., (2000) Neuroscience Letters 284: 175-1). Results also show that CH1641 gives lower molecular weight values for all three bands in comparison to the values obtained for all the other samples. These values for the CH1641 strain were closer to those obtained for BSE in sheep and, to a lesser extent, closer to bovine BSE values than those obtained for natural sheep scrapie and the SSPB1 strain. The molecular weight values for SSPB1 were indistinguishable from those obtained for natural scrapie. These are again similar findings to those published by other researchers (Hope J, et al., (1999) Gen. Vir. 80: 1-4, Baron TGM, et al., (2000) Neuroscience Letters 284: 175-178). The molecular weight for the two bovine BSE samples appeared to be closer to natural sheep scrapie and the SSPB1 molecular weights than those obtained for the Cheviot and Romney breed sheep experimentally inoculated with BSE.

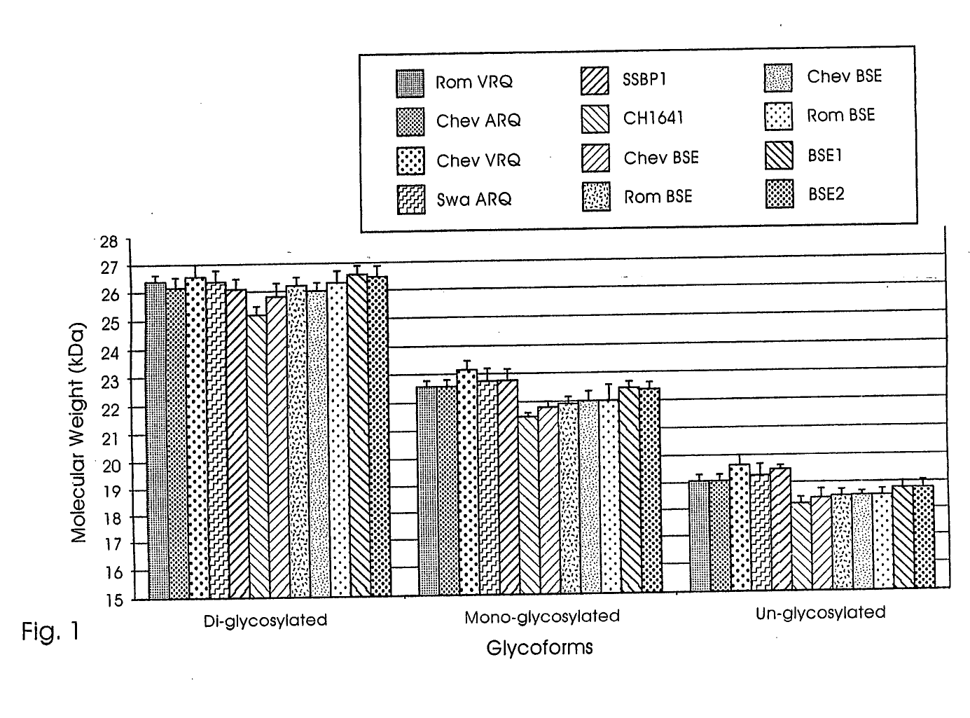

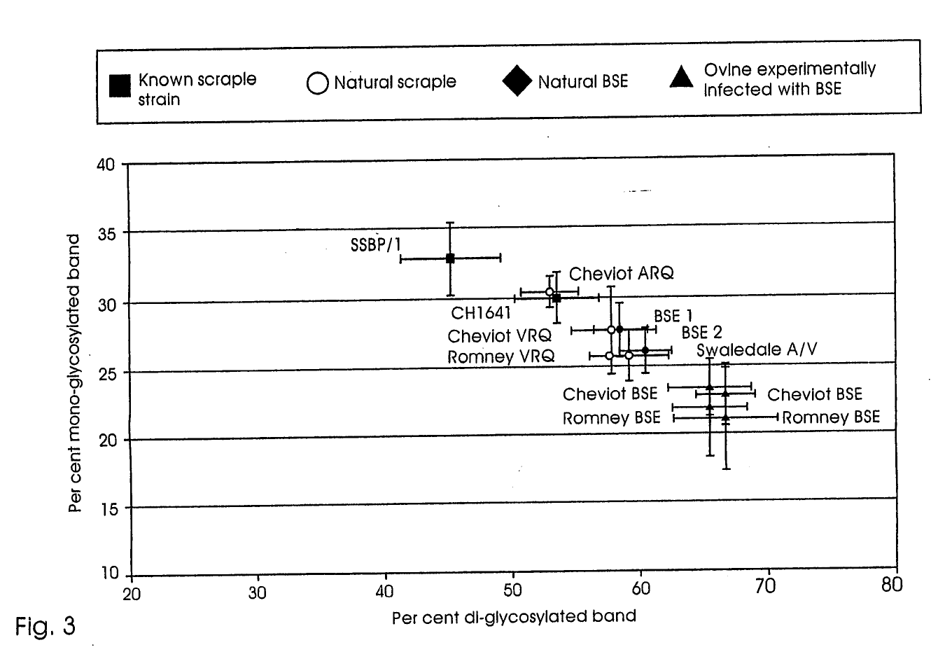

In other publications (Collinge J, et al., (1996) Nature 383: 685-690, Hill A F, et al., (1997) Nature 389, 448-450) the ratio of the different amounts of protein for each glycoform has also been used to determine strain type or species differences. Using this hybrid technique, glycoform ratios could not have been used to distinguish the natural ovine scrapie samples from the natural bovine BSE samples. However the glycoform ratios for the experimental BSE in sheep samples did appear to be positioned apart from the natural scrapie and BSE samples (FIG. 3). The glycoform ratio for SSPB1 gave the most consistently different profile to all the others whereas the CH1641 gave a glycoform profile closely resembling the profile obtained for the Cheviot ARQ/ARQ natural scrapie case. These results appear to be at odds with those obtained when molecular weight differences are used to differentiate. Observationally, the two cattle BSE cases could be differentiated from scrapie by the molecular weight differences (FIG. 1) but their positions on the glycoform ratio scattergraph (FIG. 3) were characterised by an overlap at a standard deviation from the mean.

In accordance with the invention, further differentiation can be obtained for example, by the use of the mAb P4 which appears to have a strong affinity to scrapie PrP^{Sc} and greatly reduced affinity to bovine PrP^{Sc} using this particular methodology.

The signals from the normal bovine control supplied in the Prionics test kit and the positive samples BSE1 and BSE2 were not detected by the mAb P4 antiserum using this technique. An explanation for this could be that the epitope for mAb P4 is near the amino end of the ovine PrP protein and is situated in close proximity to a proteinase K cleavage site. Using different techniques the cleavage site may not be in exactly the same place and it seems likely that an important portion of the epitope for BSE recognition can be partly or totally destroyed during the hybrid procedure, thus reducing the ability of this anti-body to bind to BSE PrP^{Sc}. If BSE in sheep has the same PrP^{Sc} conformation as BSE in cattle it is conceivable that the epitope will also be affected in the same way, although residual signal left for experimental BSE in sheep samples indicates a slightly different effect on the epitope. It could also be possible that the differences in the folding of the protein during the technique imparts differences in the conformation and this masks the epitope for BSE PrP^{Sc} and PrP^{Sc} partially masks the epitope for BSE in sheep samples, but does not have any effect on the epitope for scrapie PrP^{Sc}.

The amino acid sequence for the epitope of mAb P4 has been reported as GGGGWGQGGSHSQWNK ovine 89-104 (Harmeyer S, et al., (1998) Gen. Virol. 79: 937-945). The bovine equivalent is GGGGWGQGGTHGQWNK and differs only by 2 residues at the ovine PrP positions 98 and 100. Under the strong denaturing conditions used for immunoblotting the mAbs are considered to primarily bind linear, non-conformation-specific epitopes (Harmeyer S, et al., (1998) Gen. Virol. 79: 937-945). The total lack of affinity for bovine PrP^{Sc} and reduced affinity for BSE in sheep samples shown using the hybrid technique therefore appears to suggest differences in the structure of this epitope for BSE PrP^{Sc} and scrapie PrP^{Sc}. The differences in recognition of ovine and bovine source PrP may simply be explained by the effect of the two amino acid substitutions. However, the PrP^{Sc} extracted from BSE challenged sheep is ovine in its primary structure and the species differential no longer applies. It is however, possible that the major Proteinase K cleavage site in abnormal PrP from different strains of agent, varies under the conditions of proteolysis applied here. Thus for scrapie derived PrP^{Sc} the epitope and part of the resistant core remain intact, but for BSE derived PrP^{Sc} the major cleavage point is within or C-terminal to the mAb P4 epitope.

It has been found that the Prionics-Check test (without centrifugation steps) coupled with the mAb P4 is not very sensitive to cattle BSE PrP^{Sc}. In comparison to results using mAb 6H4 immuno-reactions are confined to a weak signal for the diglycosylated band for mAb P4 (results not shown). The hybrid technique of the present invention, and therefore the addition of the centrifugation steps appears to render the BSE PrP^{Sc} even less detectable. This maybe because centrifugation preferentially concentrates the PrP truncated C-terminally to the P4 epitope or conversely that the epitope only remains after proteolysis in small quantities as relatively soluble PrP material. It may be that the combined effects of Proteinase K digestion and the centrifugation steps on the different conformation of PrP^{Sc} are important in defining the affinity for mAbs to ruminant PrP^{Sc}.

The results presented here using the hybrid technique show differences between the bovine BSE and BSE in sheep molecular weights and glycoform ratios and whereas mAb P4 does not appear to detect the natural bovine cases at all some residual signals do occur for the BSE in sheep and the CH1641 strain. This may be due to a host factor or possible differences in protease resistance. However, the results could also indicate that the BSE PrP^{Sc} undergoes some changes in the ovine host or perhaps even gives rise to a different strain of the agent; but the latter would be contrary to our present knowledge with regard to the similar incubation period and lesion profiles in mice found for bovine BSE and sheep experimentally challenged with BSE (Bruce M, et al., (1994) Philos Trans R Soc Lond Ser B 343: 405-411). Increasing the proteinase K concentration and incubation time or decreasing the mAb P4 dilution for the hybrid technique may further reduce this residual signal.

In general, routine testing of diagnostic samples by Western immunoblotting would only be used to obtain a qualitative result but this does not give precise quantification of the differences between ovine and bovine samples. Inclusion of appropriate ovine and bovine positive controls on each gel is helpful in aiding differentiation of the BSE in sheep samples. When processed in this way the largest difference between ovine scrapie, BSE in sheep and cattle BSE was the molecular weight of the unglycosylated band but the potential for small differences in molecular weight to be easily distorted by the variable nature of electrophoresis running conditions can be problematical. This is illustrated by the fact that molecular weight values were not exactly the same for each individual sample on the eight different gels. Being able to asses this particular panel of tissues has been invaluable, as we have found that the use of the appropriate comparable controls on each gel and strict control and monitoring of all aspects of the testing is imperative for the reproducibility of the differences in molecular weight profiles and glycoform ratios. The incorporation of clearing centrifugation steps into the Prionics technique appears to give improved resolution and enhances the differences in molecular weight by giving clearer demarcation of the protein bands (results for comparison between the Prionics method and hybrid method not shown).

Interpretation of the results of the method of the invention needs to be carried out with care. An example of the difficulties encountered in analysing data is illustrated in this present study where the similarity between CH1641 and the BSE in sheep molecular weight profile, and the reduced signal that occurred for this strain when the mAb P4 was applied, may indicate the possibility of a scrapie strain origin for BSE in cattle. However, the molecular weight profile was not identical and the glycoform ratio was more like scrapie.

These results suggest that a combination of molecular weight differentiation, glycoform profiling and the two specific antibodies could be used to provide a good method of testing for BSE in the UK sheep flock.

In yet a further embodiment, the invention provides a kit for typing a strain of a transmissible spongiform encephalopathy (TSE), said kit comprising an antibody or a binding fragment thereof which binds prion protein and an antibody or a binding fragment thereof which has a different and distinguishable affinity for a particular strain of TSE, as compared to a second strain of TSE.

The invention will now be particularly described by way of example with reference to the accompanying figures in which:

FIG. 1 shows mean band molecular weights for the sample panel using the hybrid technique and mAb 6H4 antiserum. The diglycosylated bands show less molecular weight differences for the panel of samples, with the BSE in sheep (Rom BSE and Chev BSE) and the bovine BSE samples (BSE1 and BSE″) overlapping with the natural scrapie samples (Romney VRQ/VRQ, Cheviot ARQ/ARQ, Cheviot VRQ/VRQ, Swaledale ARQ/VRQ) and sheep-passaged scrapie strain SSPB1. The mono-glycosylated bands and the unglycosylated bands give almost an identical differential profile for the panel of samples, although differences between samples is greater for the unglcosylated band. There appeared to be a pattern occurring whereby CH1641 gave the lowest mean values followed by higher values for BSE in sheep, then bovine BSE, and lastly, the highest molecular weight values being those for the natural ovine scrapie samples and SSBP1.

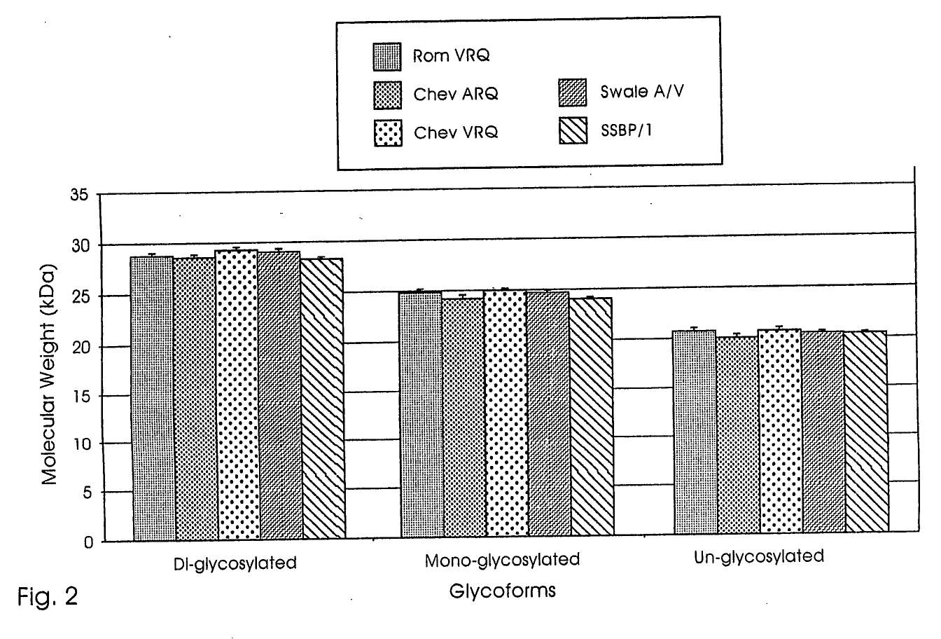

FIG. 2 shows mean band molecular weights for the natural ovine scrapie and the SSBP/1 sheep passaged scrapie sample using the hybrid technique and mAb P4. All three protein bands for the these scrapie-derived samples show very little differences giving an almost identical molecular weight profile for the panel of ovine scrapie samples (Romney VRQ/VRQ, Cheviot ARQ/ARQ, Cheviot VRQ/VRQ, Swaledale ARQ/VRQ) and the sheep-passaged strain SSBP1.

FIG. 3 is a scattergraph of the glycoform ratio of the proportion of abnormal protein in the di-glycosylated band and the mono-glycosylated band for the natural bovine BSE (BSE1 and BSE2), natural scrapie (Romney VRQ/VRQ, Cheviot VRQ/VRQ, Cheviot ARQ/VRQ, Swaledale ARQ/VRQ), the two sheeped-passaged scrapie strains (SSBP1 and CH1641) and the ovines experimentally infected with BSE (Romney ARQ/ARQ and Cheviot AHQ/AHQ. The BSE in sheep samples give unique glycoform ratios but there is considerable overlap of result between natural cases of bovine BSE and the Romney VRQ/VRQ and the Swaledale ovine scrapie. The CH1641 strain gives a closer ratio to that found for the Cheviot ARQ/ARQ scrapie sheep.

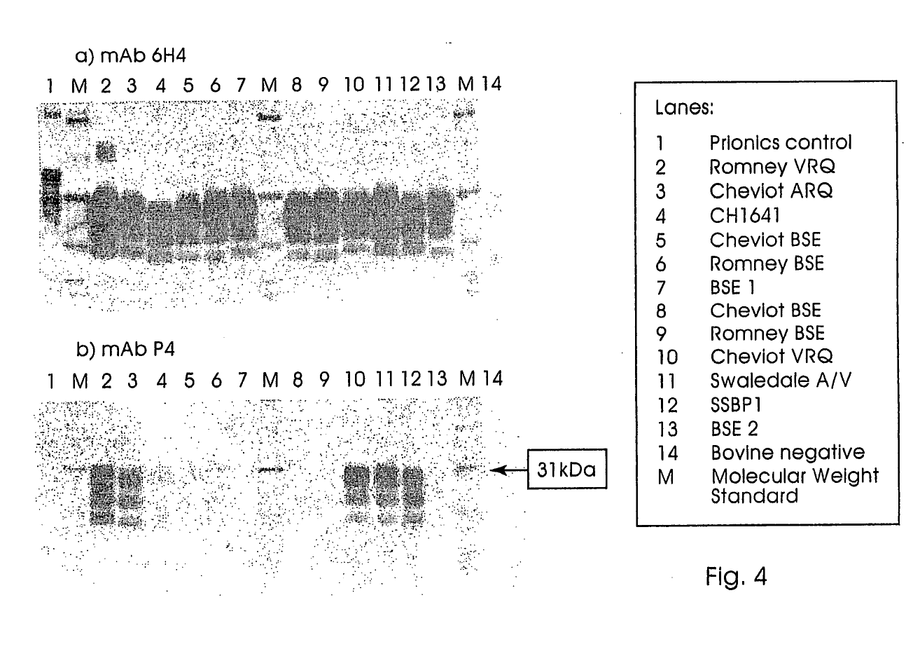

FIG. 4 shows immunoblots obtained for the panel of brain samples using the hybrid method. Membrane a) was probed with mAb 6H4, a mouse IgG1 antibody which recognises the sequence in the bovine PrP protein at the amino acid positions 144-152. Membrane b) was probed with p4 which is raised in mice, and recognises the amino acid sequence in the ovine PrP protein at amino acid positions 89-104.

Membrane a) probed with mAb 6H4 shows strong signals with both the BSE scrapie samples and the differences in molecular weights can be clearly seen.

Membrane b) probed with mAb P4 shows the strong signal with the scrapie samples (lanes 2, 3, 10 and 11) and SSBP1 (lane 12) but a reduced signal with the ovines experimentally infected with BSE (lanes 5, 6, 8, and 9) and the CH1641 strain (lane 4). The natural BSE cases (lanes 1 and 13) and the Prionics normal bovine brain control (lane 1) show no visible signal. There are very little differences in molecular weights using the mAb P4

### EXAMPLE 1

Hybrid Method for Distinguishing BSE and Scrapie in Sheep Animals and Tissues

Frozen, archived brain tissue from the brain stem region was obtained from a Romney breed sheep (AA₁₃₆RR₁₅₄QQ₁₇₁ genotype) and a Cheviot breed sheep (AA₁₃₆HH₁₅₄QQ₁₇₁ genotype), both experimentally infected with BSE. Both sheep were infected by oral dosing with 5 grams of positive bovine BSE brain material. (The sheep PrP gene produces protein of 256 amino acids, each of which is encoded by three DNA bases (one codon) in the gene. Susceptibility to scrapie has been shown to be linked to the PrP protein genotypes which are defined by variations in the amino acids encoded at codons 136, 154 and 171, and are termed polymophisms. At least five variant alleles have been found with respect to a risk of contracting scrapie which are depicted as ARQ, ARR, VRQ, AHQ and ARH. The codes respresent polymorphism of amino acids at each codon i.e. A₁₃₆R₁₅₄Q₁₇₁ (ARQ) where A=alanine, R=argenine, Q=glutamine. The other two amino acids are; H=histidine and V=valine. Homozygous and heterozygous pairing of the two alleles inherited from a ram and a ewe therefore results in considerable variation of PrP genotypes found. This level of risk varies depending on breed type and the genotypes found with the flock) [Hunter N (1997) Trends in Microbiology 5: 331-334, Dawson M, et al., (1998) Vet. Rec. 6: 623-625]. The natural scrapie brain tissues were derived from routine diagnostic submissions and comprised the following breeds and genotypes; Romney VRQ/VRQ, Cheviot VRQ/VRQ, and Cheviot ARQ/ARQ. We were unable to obtain natural scrapie samples from sheep of similar genotype to those used for the experimental BSE inoculations but breed could be matched and a Swaledale breed sheep with the heterozygous genotype ARQ/VRQ were added for further comparison.

The two BSE in cattle samples were archived tissue obtained from normal diagnostic submissions. The tissue from the sheep passaged scrapie strains, SSBP1 and CH1641 has been passaged mostly through Cheviot sheep [Wilson D R, et al., (1950) J. Comp. Pathol. 60: 267-275] and is now known to be a mixture of scrapie strains designated as A group strains [Dickinson A G, et al., (1979) Slow Transmissible Diseases of the Nervous System Vol. 1, Eds S. B. Prusiner, W. J. Hadlow. New York. Academic Press p367]. CH1641 is originally derived from a natural case of scrapie in a Cheviot [Dickinson A G, et al., (1986) Unconventional Viruses and Central Nervous System Diseases, Part III chapter 9 446-460 Eds. L. Court.D. Dormont. D. Kingsbury. Moisdon la Riviere, Abbaye de Mellaray] and has been characterised by serial passage in sheep as either a single strain or an unresolved mixture of strains. It has unusual changes in incubation properties on the second and third passage in comparison to Group A strains and has been classified as a C Group strain [Foster J D, et al., (1988) Vet. Rec. 123: 5-8, Dickinson A G, et al., (1988) Novel Infectious Agents and the Central Nervous System. Ciba Foundation Symposium No. 135. Eds G.Bock, J. Marsh, Chichester, Wiley p63]

Western Immunoblotting Technique

A hybrid technique, which was a modified Prionics based technique [Schaller O, et al., (1999) Acta. Neuropathol. 98: 437-443] incorporating centrifugation steps [Collinge J, et al., (1996) Nature 383: 685-690], was used to detect PrP^{Sc}.

Essentially, 1.5 μl of a 10% homogenate (prionics) was centrifuged at 1127 g for 5 minutes (TLA 45 rotor-Beckman). Proteinase K (Roache) (5 μl of a 1 mg/ml stock to give a final concentration of 50 μg/ml) was added to 100 ml of the supernatant and this was incubated at 37° C. for 1 h. Pefabloc (Boehringer to 1 mM) and 100 ml of sample buffer (Prionics) was added and incubated at 100° C. for 10 min. Centrifugation was carried out in a microcentifuge (14,000 rpm for 5 min) and 10 μl of the supernatant was loaded onto 12% Bis-Tris polyacrylamide gels (Invitrogen). Electorphoresis was carried out at 200 v for 35 min and Western immunoblotting on to polyvinylidene difluoride PVDF membrane (Millipore) at 150 v for 1 h. The blots were blocked in 50 ml of blocking buffer (Prionics) for 1 h and incubated over night at 4° C. in a 1:5000 dilution of primary antibody (6H4 Prionics in blocking buffer). Membranes were washed in TBS (with 0.05% Tween 20) 4×7 min and incubated secondary antibody (1:5000) (goat anti-mouse conjugated to alkaline phosphatase) (Prionics) for 1 hour at room temperature. They were then washed again in TBST 4×7 min and then incubated in luminescence buffer (Prionics) for 5 min. The labelling was visualised by means of enchanced chemiluminescence system (CPD-Star Tropix). Signals were quantified using Fluor S Multimager computer analysis (Quantity One software, Biorad UK Ltd). Using this system, molecular weights were measured by comparison to the biotinylated markers on the gel and the centre position for each sample band is recorded as the molecular weight. For glycoform analysis the centre position is again used as the reading point. The combined signals were defined as 100% and the contribution of each band calculated as a percentage of the whole.

Antisera

For monoclonal antibody (mAb) P4 experiments, this antiserum was substituted for the mAb 6H4 and used at a dilution of 1:2500. The mAb 6H4 is a mouse IgG1 antibody which recognises the sequence in the bovine PrP protein at the amino acid positions 144-152 [Korth C, et al., (1997) Nature 390: 74-77]. The mAb P4 was raised in mice, and recognises the amino acid sequence in the ovine PrP protein amino acid positions 89-104. [Harmeyer S, et al., (1998) Gen. Virol. 79: 937-945].

Gel Plan

Seventeen wells were set up on each gel,-Lane 1 contained the Prionics control sample consisting of a mixture of molecular weight markers and normal bovine brain tissue and, in the interests of the accuracy of subsequent molecular weight measurements, three of the wells contained the biotinylated molecular weight marker (Lanes 2, 9 and 14). The rest of the gel was set up from left to right as follows:—Romney VRQ/VRQ natural scrapie, Cheviot ARQ/ARQ natural scrapie, CH1641, Cheviot BSE in sheep, Romney BSE in sheep, Bovine BSE 1, duplicate sample of the Cheviot BSE in sheep, duplicate sample of the Romney BSE in sheep, Cheviot VRQ/VRQ natural scrapie, Swaledale ARQ/VRQ natural scrapie, SSBP1, bovine BSE 2 and a normal bovine negative. Eight repeats of the gel were processed using the same homogenates for each of the antisera, 16 gels in all. The gel plan was the same whether the antiserum used was mAb 6H4 or the mAb P4.

Results

Molecular Weight Analysis

All samples gave a characteristic protein banding pattern corresponding to the three glycoforms of PrP^{Sc}, with the di-glycosylated, (top band) showing the strongest signal, the mono-glycosylated (middle band) having the next strongest signal followed by the unglycosylated (bottom band) having the weakest signal. The mean molecular weights and standard deviation of all three bands obtained for the panel of samples from the eight repeats using the mAb 6H4 antiserum are shown as a histogram in FIG. 1. For the mono-glycosylated bands and the unglycosylated band there appeared to be a pattern occurring whereby CH1641 gave the lowest mean molecular weight values followed by higher values for BSE in sheep, then bovine BSE and lastly, the highest molecular values being those for the natural ovine scrapie samples and SSBP1. This overall pattern of results for the panel of samples was similar for the mono-glycosylated and unglycosylated protein band the differences were greater for the unglycosylated band. The di-glycosylated bands could not be used to differentiate the panel of samples by molecular weight.

The molecular weights obtained for the CH1641 scrapie strain gave consistently lower molecular weight measurements for all three bands than all the other samples but values were closer to the BSE in sheep and bovine BSE molecular weights than those obtained for the scrapie and the SSPB1 samples. The SSBP1 molecular weights were considered to be indistinguishable from those obtained for the natural scrapie samples.

The mean molecular weights and standard deviation obtained for the protein bands from the eight runs for each sample which gave detectable signals using the mAb P4 antiserum (the natural sheep scrapie samples and the SSBP1) are shown in FIG. 2. The molecular weight profiles were similar for all of these scrapie samples but molecular weights of all three bands were higher than those obtained with mAb 6H4.

Glycoform Ratios

With regard to the glycoform ratios, obtained with the mAb 6H4 antiserum, the ratio of the mean values of the high molecular mass glycoform (di-glycosylated band) and the low molecular mass glycoform (mono-glycosylated band) were plotted as a scattergraph (FIG. 3). The SSBP1 had a ratio which appeared to stand apart from the others (45:32). The CH1641 ratio (53:29) was very similar to that obtained for the Cheviot ARQ/ARQ (52:30) natural scrapie sample. The Cheviot VRQ/VRQ, (57:27) Romney VRQ/VRQ (57:25) and Swaldale ARQ/VRQ (58:25) natural scarpie samples gave ratios which were similar to that obtained for one of the cattle BSE samples (60:26). The other cattle BSE sample had a ratio closer to the BSE in sheep samples, with regard to its lower molecular mass (59:22). The ratios for the two duplicate samples from the Cheviot experimentally inoculated with BSE were 65:23 and 66:23. The ratios for the two duplicate samples from the Romney experimentally incolulated with BSE were 65:22 and 66:21. Standard deviations for the glycoform ratios were varied for the eight runs with the BSE in cattle tending to overlap with the Cheviot VRQ/VRQ, Romney VRQ/VRQ and Swaldale ARQ/VRQ scrapie samples. However, the CH1641 and Cheviot ARQ/ARQ ratios had little overlap and the BSE in sheep and SSBP1 ratios did not appear to overlap with any of the other glycoform ratios.

The mean glycoform ratios obtained using mAb P4 were generally higher than those obtained for the natural sheep samples using the mAb 6H4 antiserum; SSBP1 (46:31), Cheviot ARQ/ARQ (57:27), Cheviot VRQ/VRQ, (62:23) Romney VRQ/VRQ (59:25) and Swaldale ARQ/VRQ (62:24). None of these were similar to the BSE in sheep glycoform ratios found using the mAb 6H4.

Differentiation Using mAb 6H4 and mAb P4

Two representative gel results for the panel of samples are shown in FIG. 4. The top row of immunoblots shows the results using the hybrid technique of the invention and the mAb 6H4 antiserum and the bottom row the results using the mAb P4 antiserum. Observations when the mAb P4 was used were that all ovine scrapie samples and the SSBP1 gave strongly stained bands. The CH1641 and BSE in sheep samples had greatly reduced signals for all three bands and no signal at all could be detected for the bovine BSE samples. Also, the signal for the normal bovine brain sample supplied as the Prionics control was also undetected. Exactly the same results were found for the eight repeats of the gel.

1. A method for typing a strain of a transmissible spongiform encephalopathy (TSE) in an infected animal, said method comprising
a) separating a sample of abnormal prion protein on the basis of molecular weight and/or glycoform ratios, and detecting the separated forms;
b) contacting the sample with an antibody or a binding fragment thereof which binds prion protein from a strain of TSE as found in the sample with a different and distinguishable binding affinity to that of at least one other strain of TSE, and detecting bound antibody or binding fragment; and
c) using the results of (a) and (b) to determine the type of TSE strain present in the sample.

2. A method according to claim 1 wherein in step (b) the presence of a particular sequence is detected using an antibody or a binding fragment thereof which has different affinities for a peptide sequence which constitutes an epitopic region of a prion protein which includes some differences in different strains.

3. A method according to claim 2 for detecting the difference between BSE and scrapie, wherein the antibody is an antibody raised to a peptide corresponding to amino acids 89-104 of ovine spongiform encephalopathy or an epitopic region thereof.

4. A method according to claim 1 which is carried out on an extract of brain tissue which has been separated on the basis of molecular weight, and detected using an antibody or a binding fragment thereof which is specific for a sequence which appears in prion protein.

5. A method according to claim 4 wherein the separation is on an electrophoretic gel.

6. A method according to claim 1 wherein differences in the molecular weights of diglycosylated, monoglycosylated or unglycosylated forms of abnormal prion protein is detected and used as a further method of differentiation between strains.

7. A method according to claim 6 wherein the molecular weight of the unglycosylated protein is used as a means of distinguishing between strains.

8. A method according to claim 1 wherein the ratio of diglycosylated, monoglycosylated or unglycosylated forms of abnormal prion protein is detected and used as a further method of differentiation between strains.

9. A method according to claim 1 wherein both molecular weight differentiation and glycoform profiling are effected prior to step (b).

10. A method according to claim 1 for detecting BSE in sheep.

11. A method according to claim 1 which comprises the step of centrifuging a sample of homogenised tissue from an animal suspected of having a TSE, subjecting the product to an enzyme which digests normal protein, but to which abnormal prion protein is resistant, separating the thus formed mixture on a gel, probing the separated mixture with (i) an antibody which binds a prion peptide, and (ii) antibody which has strong affinity for some prion peptides and weaker affinity for others, and typing the strain of TSE on the basis of the characteristics of the signals produced.

12. A method according to claim 11 for detecting BSE in sheep, wherein the antibody used in step (i) is an antibody which binds the bovine PrP protein at the amino acid positions 144-152 and the antibody used in step (ii) is an antibody which recognises the amino acid sequence in the ovine PrP protein amino acid positions 89-104.

13. A kit for typing a strain of a transmissible spongiform encephalopathy (TSE) using a method according to claim 1, said kit comprising (a) an antibody or a binding fragment thereof which binds prion protein and (b) an antibody or a binding fragment thereof which has a different and distinguishable affinity for a particular strain of TSE, as compared to a second strain of TSE.

14. A kit according to claim 13 wherein the antibody of (a) is an antibody which recognises bovine PrP protein at the amino acid positions 144-152.

15. A kit according to claim 13 wherein the antibody of (b) is an antibody which recognises the amino acid sequence in the ovine PrP protein at amino acid positions 89-104.

16. A method for typing a strain of a transmissible spongiform encephalopathy (TSE) in an infected animal substantially as hereinbefore described.
